(19) United States
(12) Patent Application Publication
(10) Pub. No.: US 2020/0054581 A1
(43) Pub. Date: Feb. 20, 2020

(54) APPLICATION OF TRPM7 INHIBITORS TO TREAT SLEEP APNEA AND HYPERTENSION IN OBESITY

(71) Applicant: THE JOHNS HOPKINS UNIVERSITY, Baltimore, MD (US)

(72) Inventors: Vsevolod Polotsky, Pikesville, MD (US); James Sham, Ellicott City, MD (US); Wan-Yee Tang, Baltimore, MD (US)

(21) Appl. No.: 16/487,143

(22) PCT Filed: Feb. 20, 2018
(86) PCT No.: PCT/US2018/018667

§ 371 Date: Aug. 20, 2019

Related U.S. Application Data
(60) Provisional application No. 62/461,452, filed on Feb. 21, 2017.

(51) Int. Cl. A61K 31/137; A61K 31/4174; A61K 31/4184; A61K 31/69; A61P 11/00; A61P 9/00

(52) U.S. Cl. CPC A61K 31/137; A61K 31/4174; A61P 9/00; A61K 31/69; A61P 11/00; A61K 31/4184

(57) ABSTRACT

Methods for prevention or treatment of a respiratory disease and a cardiovascular disease in subjects comprising administering to the obese subject an effective amount of TRPM7 inhibitor.

### REFERENCE TO RELATED APPLICATIONS

This application claims the benefit of U.S. Provisional Patent application 62/461,452 filed Feb. 21, 2017, which is hereby incorporated by reference for all purposes as if fully set forth herein.

### STATEMENT OF GOVERNMENTAL INTEREST

This invention was made with government support under grant no. HL133100, awarded by the National Institutes of Health. The government has certain rights in the invention.

### BACKGROUND OF THE INVENTION

Obesity is a highly prevalent condition observed in 34.9% of US adults. Obesity leads to cardiovascular disease1 increasing mortality by 2-3 fold. Excessive adiposity causes multiple complications including obstructive sleep apnea (OSA) and hypertension, which greatly contribute to the cardiovascular risk. OSA is recurrent upper airway obstruction during sleep leading to intermittent hypoxia and sleep fragmentation. Mechanisms of OSA in obesity are related to the anatomically compromised upper airway and other factors, including pathologically increased hypoxic ventilatory responses, which leads to respiratory instability, i.e. high loop gain. Nasal continuous positive airway pressure (CPAP) relieves OSA, but does not modify physiological traits of sleep apnea. In addition, poor adherence to CPAP severely limits its use. There is no pharmacotherapy for OSA. The high prevalence of hypertension and resistant hypertension in obesity has been linked to SNS activation related to obesity per se and to comorbid OSA. However, CPAP improves control of blood pressure only in 25-30% of adherent patients. Moreover, greater than 20% of all hypertensive patients adherent to therapy are resistant to the optimal medical regimen with obesity as a key risk factor. Thus, treatment of OSA and hypertension in obesity poses significant therapeutic challenges and new treatment modalities are urgently needed.

### SUMMARY OF THE INVENTION

One embodiment of the present invention is a method for the prevention or treatment of a respiratory disease, a cardiovascular disease, or both in an obese subject comprising administering to the obese subject an effective amount of TRPM7 inhibitor or a pharmaceutically acceptable salt, solvate, or stereoisomer thereof. Examples of a respiratory disease may be sleep apnea, obstructive sleep apnea, central apnea, or Cheyne-Stokes respiration. Examples of a cardiovascular disease maybe hypertension or heart failure. The method may be applied to subjects including humans or animals. It is preferred that the TRPM7 inhibitor is a pharmaceutical composition comprising a pharmaceutically acceptable carrier. Examples of TRPM7 inhibitors that may be used in the present invention include 2-APB 174, Spermine, SKF-96365, Nafamostat, Carvacrol, NDGA, AA861, MK886, Waixenicin A, NS8593, Quinine, CyPPA, Dequalinium, SKA31, UCL 1684, Sphingosine, fingolimod, and combinations thereof. The preferred TRPM7 inhibitor is fingolimod. The TRPM7 inhibitor may be administered by a topical, oral, or injection means.

Another embodiment of the present invention is a method for prevention or treatment of a cardiovascular disease in a subject comprising administering to the subject an effective amount of TRPM7 inhibitor or a pharmaceutically acceptable salt, solvate, or stereoisomer thereof. Examples of a cardiovascular disease may be hypertension or heart failure and the subject may be a human or an animal. Typically, the subject is obese. It is preferred that theTRPM7 inhibitor is a pharmaceutical composition comprising a pharmaceutically acceptable carrier. Examples of TRPM7 inhibitors that may be used in the present invention include 2-APB 174, Spermine, SKF-96365, Nafamostat, Carvacrol, NDGA, AA861, MK886, Waixenicin A, NS8593, Quinine, CyPPA, Dequalinium, SKA31, UCL 1684, Sphingosine, fingolimod and combinations thereof. The TRPM7 inhibitor may be administered by a topical, oral, or injection means.

Another embodiment of the present invention is a method for prevention or treatment of a respiratory disease in a subject comprising administering to the subject an effective amount of TRPM7 inhibitor or a pharmaceutically acceptable salt, solvate, or stereoisomer thereof. Examples of a respiratory disease may be sleep apnea and obstructive sleep apnea. The method may be applied to subjects including humans or animals. It is preferred that the TRPM7 inhibitor is a pharmaceutical composition comprising a pharmaceutically acceptable carrier. Examples of TRPM7 inhibitors that may be used in the present invention include 2-APB 174, Spermine, SKF-96365, Nafamostat, Carvacrol, NDGA, AA861, MK886, Waixenicin A, NS8593, Quinine, CyPPA, Dequalinium, SKA31, UCL 1684, Sphingosine, fingolimod, and combinations thereof. The preferred TRPM7 inhibitor is fingolimod. The TRPM7 inhibitor may be administered by a topical, oral, or injection means. The method of claim 21 wherein the TRPM7 inhibitors are selected from the group consisting of 2-APB 174, Spermine, SKF-96365, Nafamostat, Carvacrol, NDGA, AA861, MK886, Waixenicin A, NS8593, Quinine, CyPPA, Dequalinium, SKA31, UCL 1684, Sphingosine, fingolimod, and combinations thereof.

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “disease” is meant any condition or disorder that damages or interferes with the normal function of a cell, tissue, or organ. Examples of diseases include sleep apnea and/or hypertension, as examples.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The term “inhibitor” means to inhibit the expression (i.e. transcription, translation) of TRPM7 or the activity of the TRPM7 protein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “reduces” is meant a negative alteration of at least 10%, 25%, 50%, 75%, or 100%.

By “reference” is meant a standard or control condition.

By “subject” is meant a mammal, including, but not limited to, a human or non-human mammal, such as a murine, bovine, equine, canine, ovine, or feline.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

The term “TRPM7” denotes a protein.

The term “Trpm7” denotes a gene.

### BRIEF DESCRIPTION OF THE DRAWINGS

FIG. 1 illustrates the a putative mechanism by which obesity acts on the carotid body to increase blood pressure and exacerbate obstructive sleep apnea (OSA). ObRb, Leptin ObRb receptor; TRPM7, transient receptor potential melastatin 7; CSN, carotid sinus nerve; HVR, hypoxic ventilatory response; SNS, sympathetic nervous system, HTN, hypertension.

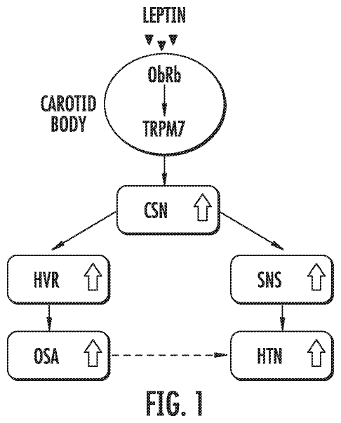

FIG. 2 illustrates colocalization of ObRb receptor (red) and Trpm7 (magenta) in tyrosine hydroxylase (green) positive glomus cells of C57BL/6J mouse by immunostaining. Blue staining designates nuclei.

FIG. 3 illustrates Leptin augments hypoxia induced CSN activity in isolated superfused CB-CSN preparation (left, top trace). The effect of leptin was dampened by TRP blockers SKF96365 (SKF) (left, bottom trace). Right panel shows the summary data of the hypoxia induced change in CSN activity in the absence and presence of leptin. SKF and 2-APB data were pooled, *p<0.05.

FIGS. 4A and 4B illustrates preferential expression of Trpm7 in CB of WT mice, and its down-regulation in ob/ob and db/db mice. (A) Transcriptome array analysis revealed the expression level of TRPs genes as quantified by the biotin intensity of probes in the microarray. ND: Not detected. (B) mRNA expression level of Trpm3, Trpm6 and Trpm7 in brain, CB, PG and SCG from WT, db/db and ob/ob mice relative to WT brain samples. The 2-ΔΔCt method was used to calculate the relative transcript level (RER) which normalized to Rpl19. Values are mean±SEM (n=2-4). *p<0.05 vs WT mice.

FIG. 5A-5C illustrates leptin enhances TRPM7 current in leptin receptor expressing PC12 cells. (A) Time course changes in non-selective cation current (at 100 mV recorded by a ramp protocol from −100 to +100 mV activated by the TRPM7 agonists naltriben and leptin. a-f indicate where traces were selected for display in B and C. (B) Current-voltage relations recorded before (a), during (b) and after (c) naltriben application. (C) Current-voltage relations recorded before (c) and during leptin application (d). The TRPM7 antagonist NS8593 completely blocked the current (e), which was partially recovered after washout (f).

FIG. 6A-6C is a similar experiment as described in FIG. 5 showing leptin enhanced non-selective cation current was significantly inhibited by the TRPM7 antagonist FTY720 is used instead of NS8593.

FIG. 7 illustrates leptin receptor (ObRb) signaling pathway and the potential mechanisms of Trpm7 regulation in glomus cells.

FIG. 8A-8C illustrates exposure of leptin to Leprb-expressing PC12^{LEPRb} cells increased Trpm7 promoter activity and gene transcription. (A) PC12^{LEPRb} cells transfected with either pEZX-Luc-vector (empty, no insert) or pEZX-Luc-Trpm7 (promoter insert) were exposed to 1 ng/ml leptin for 48 hr prior to measurement of luciferase activity. B and C) Gene expression and promoter methylation of Trpm7 in cells exposed to leptin or 5-aza-dC for 48 hr. Trpm7 mRNA level was quantitated by qPCR. Methylation status of CpG sites in Trpm7 promoter were determined by bisulfite sequencing. A total of 40 CpG sites per clone and 6 individual clones for each treatment group were sequenced. Each row of circles represented an individual clone. Putative TF binding sites such as STAT3 and NFkB were shown in scale in Trpm7 promoter. TSS: transcription start site. Boxed regions highlight the CpG sites showing differential methylation in cells exposed to leptin or 5-aza-dC. (p<0.05). Values were mean±SD.

FIGS. 9A and 9B illustrates decrease in Trpm7 gene expression and increase in Trpm7 promoter methylation in CB from db/db and ob/ob mice. (A) Trpm7 mRNA levels quantified by qPCR and (B) percent methylation of Trpm7 sites assayed by bisulfite sequencing. *p<0.05 or **p<0.01 compared to WT mice. Values were mean±SEM (n=2-4 per group).

FIG. 10 illustrates leptin infusion increased minute ventilation (V_{E}) and the hypoxic ventilatory response (HVR) in C5BL/6J mice. *p<0.05 for the effect of leptin. Ł and [symbol] , p<0.01 and <0.001 for the effect of hypoxia.

FIG. 11 illustrates carotid body transfection with Ad-Lepr^{b} carrying the ObRb gene dramatically increased ObRb (A) and Trpm7 (B) mRNA expression in the carotid body (CB) of leptin receptor deficient db/db mice, but had no effect in the hypothalamus and medulla. Relative Expression Ratio (RER) values were calculated by 2-ddCt method, where the RER was set as 1.00 in mice transfected with Ad-LacZ control vector. Error bars: SEM. **, p<0.001 vs Ad-LacZ (control); *p<0.05 vs WT (Ad-LacZ)

FIG. 12 illustrates overexpression of the leptin ObRb receptor (Ad-Lepr^{b}) in the carotid bodies of leptin receptor deficient obese db/db mice increases minute ventilation (V_{E}, panel A) only during acute hypoxia (10% O₂) challenge increasing the hypoxic ventilatory response (HVR, panel B), which does not occur in mice infected with control (Ad-LacZ). *p<0.05 for the effect of Ad-Lepr^{b} compared to Day 0 and to Ad-LacZ; ł and [symbol] , p<0.01 and <0.001 for the effect of hypoxia; Panel C, Representative respiratory tracings in the db/db mouse after transfection with Ad-LacZ (control virus) vs Ad-Lepr^{b} (ObRb receptor) show hyperventilation in the ObRb transfected mouse only in 10% O₂, but not at baseline.

FIG. 13 illustrates an obstructive hypopnea during REM sleep in db/db mouse (RIGHT PANEL). The shaded area is expanded on the LEFT PANEL. * denotes inspiratory flow limitation.

FIG. 14 illustrates leptin-induced increases in minute ventilation (V_{E}) during acute hypoxia and the hypoxic ventilatory response (HVR) were abolished by a TRPM7 blocker NS8593.

FIG. 15 illustrates the experimental design of prelim exp. 3: CSND, carotid sinus nerve dissection (CB denervation).

FIG. 16A-C, A illustrates leptin increases mean arterial pressure and heart rate and the effects of leptin are abolished by carotid sinus nerve dissection (CSND). *p<0.001 compared to all other conditions. B illustrates that overexpression of the leptin ObRb receptor (Ad-Lepr^{b}) in the carotid bodies of leptin receptor deficient obese db/db mice increases minute ventilation compared to the Ad-luc control. C illustrates that leptin-induced hypertension was abolished by subcutaneous and administration of FTY720.

### DETAILED DESCRIPTION OF THE INVENTION

The present invention are methods for the prevention or treatment of a respiratory disease and a cardiovascular disease in an subjects having one or more of these diseases through the administration of one or more TRPM7 inhibitor(s) Through the study of elevated leptin levels in obese patients the inventors of the present invention identified a critical role of TRPM7 in disease.

Obesity leads to high cardiovascular morbidity and mortality acting via multiple mechanisms including increased prevalence and severity of hypertension and obstructive sleep apnea (OSA). The inventors of this invention first developed a thesis that obesity exacerbates hypertension and OSA via increased levels of leptin, an adipocyte-produced hormone. Leptin suppresses appetite and increases metabolic rate and thus leptin deficient ob/ob and leptin receptor (ObRb or Lepr^{b}) deficient db/db mice are obese and hypometabolic. However, obese humans have high leptin plasma levels, which are increased in proportion to the adipose mass. Resistance to metabolic effects of leptin is caused by limited permeability of the blood-brain barrier (BBB) and impaired leptin ObRb receptor signaling. Leptin is also a potent stimulator of the sympathetic nervous system (SNS), and hyperleptinemia is associated with hypertension in obese humans and rodents. Obesity heightens the HVR resulting in respiratory instability exacerbating comorbid OSA and hypertension. Thus, leptin contributes to the pathogenesis of hypertension and OSA in obesity, but mechanisms are not clear.

CB are major peripheral hypoxia sensors transmitting chemosensory input via the carotid sinus nerve (CSN) to the medullary centers, which results in acute hyperventilation in response to hypoxia and the activation of the SNS. Obesity and comorbid OSA sensitize the CB. ObRb, the longest isoform of leptin receptors primarily responsible for leptin signaling, is abundantly expressed in CB, but the significance of leptin signaling in CB is unknown. The inventors discovered, based on the in vitro preliminary data (described below), that (1) leptin activates CB via non-selective cation transient receptor potential channels (TRP); (2) the hypoxia-sensitive transient receptor potential melastatin 7 gene (Trpm7) is predominantly and preferentially expressed in the CB; and (3) leptin stimulates TRPM7 current, upregulates Trpm7 expression, and induces Trpm7 promoter activity and demethylation. The in vivo data described below also led the inventors to discover that 1) leptin regulates the HVR and this effect is abolished by TRPM7 blockers; 2) leptin induces hypertension and this effect is abolished by CSN denervation; 3) leptin or ObRb deficiency is associated with reduced expression of Trpm7 in CB; and 4) replenishment of ObRb in CB of db/db mice increases Trpm7 expression and the HVR exacerbating sleep apnea. The data led to the conclusion that leptin augments the CB chemoreflex acting via TRPM7 channels to exacerbate OSA and induce hypertension and, therefore, TRPM7 blockers administered to the CB area will treat obesity-induced OSA and hypertension. The present invention identifies a novel therapeutic target for OSA and hypertension and sleep apnea and the following therapeutic agents 2-APB 174, Spermine, SKF-96365, Nafamostat, Carvacrol, NDGA, AA861, MK886, Waixenicin A, NS8593, Quinine, CyPPA, Dequalinium, SKA31, UCL 1684, Sphingosine, FTY720 and combinations thereof, which are inhibitors of TRPM7.

Obesity leads to high levels of adipokine leptin. Increased leptin levels have been previously reported in OSA and implicated in increased SNS activity and the pathogenesis of hypertension. However, mechanisms of the effects of leptin on blood pressure and sleep apnea are unclear. The significance of the present invention is establishing the novel concept that leptin signaling in the carotid body (CB) is crucial for the pathogenesis of hypertension and sleep apnea and serum leptin levels may serve as a biomarker of resistant hypertension and OSA in obese patients. However, leptin receptor per se cannot be used as a therapeutic target, because of potential adverse effects of leptin resistance on numerous metabolic outcomes, including obesity and diabetes. Therefore, it is of particular significance that we identified TRPM7 channel as a downstream pathway for the effects of leptin on the CB chemoreflex and SNS. TRPM7 inhibitors are available and have been successfully used in rodents and humans. The present invention is the use of TRPM7 inhibitors as a fundamentally new treatment of hypertension and sleep apnea in obesity. Furthermore, systemic (oral or injection) or topical applications of TRPM7 inhibitors may be considered for treatment of other conditions with the hyperactive ventilatory drive, e.g. central apnea and Cheyne-Stokes respiration, or increased SNS activity, e.g. heart failure. Notably, CB ablation has previously been explored for treatment of heart failure. In summary, the clinical significance of our proposal may reach beyond treatment of complications of obesity.

In addition, the present invention opens new doors for the studies on (1) hormonal modulation of CB functions; (2) a new candidate channel in CB chemoreception; and (3) the epigenetic regulation of CB functions and control of breathing. In particular, epigenetic modification of gene expression persists over time. Alterations in the methylation status of Trpm7 and perhaps other genes in CB by hormones, environmental conditions or nutritional status (e.g. starvation) may predispose CB to aberrant cardiopulmonary regulation. The present invention, highlights CB pathology as an important but as yet under-recognized mechanism, which may lead to a shift in paradigm in our understanding of respiratory control.

First, the present invention identified that hyperleptinemia causes hypertension by acting on peripheral sensors in the CB. Poor permeability of the blood-brain barrier (BBB) is one of the major mechanisms of leptin resistance, occurring in the hypothalamus and medulla, for food intake and metabolic rate. The present invention identified that leptin-resistance is selective for the CNS, and the peripheral actions of leptin can escape leptin-resistance and exert its effects on SNS activity and blood pressure. Second, the present invention identifies that peripheral effects of leptin on the CB chemoreflex induce respiratory instability and aggravate sleep apnea. The stimulating effect of leptin on the medullary respiratory centers protects against alveolar hypoventilation in obesity, but the majority of obese patients are resistant to the central effects of leptin because of the BBB limitations. The present invention implicates the peripheral effects of leptin in the pathogenesis of abnormally high loop gain and sleep apnea. Third, the present invention identifies that leptin modulates CB function via acute activation and chronic upregulation of TRPM7. The data generated during the creation of the present invention identifies, for the first time, that TRPM7 is a downstream target of the leptin-signaling cascade and that it may contribute to CB chemoreception. The acute activation of TRPM channel activity and chronic upregulation of Trpm7 transcription, in part through epigenetic modulation, may provide a unique mechanism for the control of CB functions.

The present invention successfully utilized a novel technique of applying Ad-Lepr^{b} adenovirus suspension in Matrigel to locally express ObRb in CB without affecting ObRb expression in the brainstem and hypothalamus of leptin receptor deficient db/db mice. This novel Matrigel approach was applied to create ObRb and Trpm7 knockouts in the CB by infecting ObRb and Trpm7 floxed mice, respectively, with the Ad-Cre-GFP virus in the CB region to avoid complications due to ObRb and Trpm7 deletion in the global KO mice. Epigenetic techniques including bisulfile sequencing with modified procedures for small samples were applied to assess methylation in specific CpG sites of Trpm7 promoter in mouse CB. These approaches together with the special technique of patch-clamping glomus cells in whole carotid body preparation, the state-of-the-art techniques for continuous recording of sleep apnea and telemetric measurement were used.

### In Vitro Experiments

The inventors performed experiments to demonstrate: 1) that leptin enhances CB output via modulation of TRPM7 channel activity and its gene expression, 2) to determine the contributions of TRPM7 channels to the enhance CSN activity, membrane depolarization and increase [Ca²⁺]ᵢ in glomus cells, 3) delineate the specific signaling pathways activated acutely by leptin, and 4) to identify the specific transcriptions factors and the epigenetic mechanisms responsible for Trpm7 upregulation induced by prolonged leptin exposure.

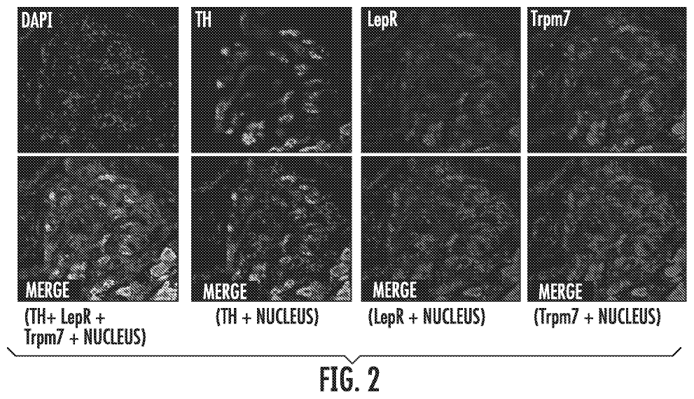

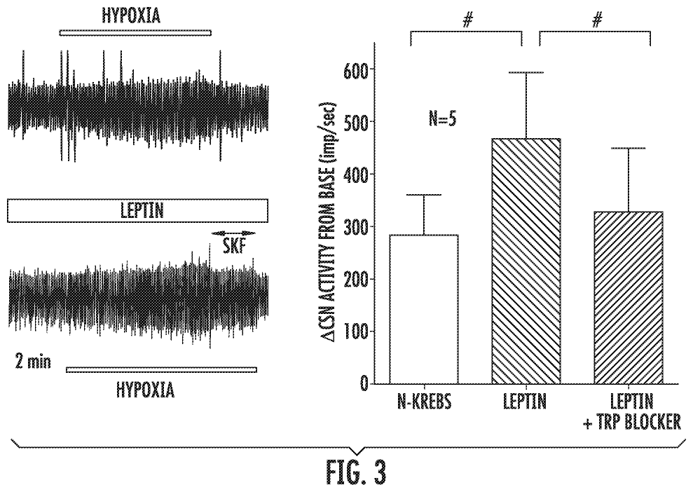

Leptin and carotid body function CBs are major peripheral O₂ sensors, which initiate chemosensory inputs in the CSN to the respiratory centers via nucleus solitarius to evoke acute hyperventilation, and the rostral ventrolateral medulla presympathetic neurons to stimulate SNS activity. CB plays critical roles in the development of hypertension during chronic intermittent hypoxia (CIH); and the sympatho-excitation and destabilization of breathing during heart failure. However, the connection between CB and the obesity-associated hypertension and respiratory instability has not been clearly established. Previous studies showed that the leptin ObRb receptor is abundantly expressed in the glomus cells in CB of rat and human and its expression is altered by CIH. The inventors also detected ObRb and Trpm7, which were co-localized in the tyrosine hypdroxylase positive glomus cells of C57BL/6J mouse (FIG. 2). The effect of leptin on isolated CB-CSN preparations from C57B1/6J (WT) mice was studied. The inventors found that leptin significantly augmented hypoxia-induced CSN activity; and the enhanced CSN activity was blocked by the nonspecific TRP channel antagonists SKF96365 and 2-aminoethoxydiphenylborane (2-APB) (FIG. 3). These observations clearly suggested to the inventors that leptin and ObRb receptors are related to CB function, and leptin may modulate CB activity through regulation of TRP channels.

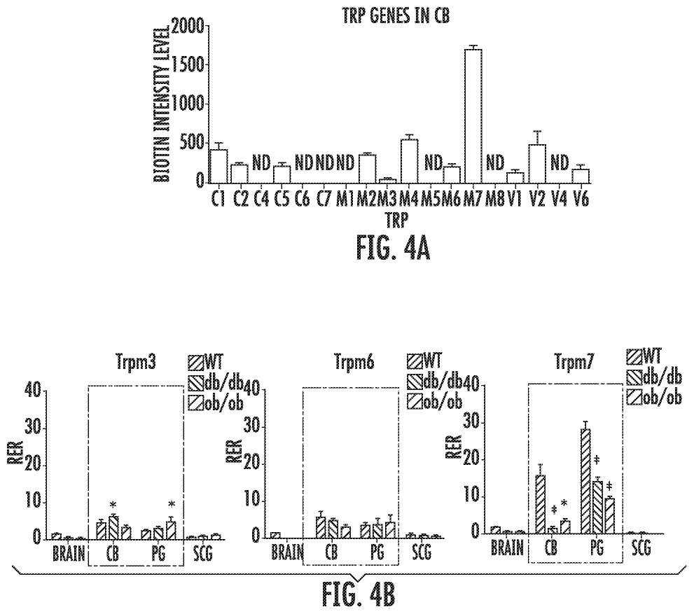

Carotid body and TRP channels. It is well documented that hypoxia activates CB by inhibition of K⁺ channels (including voltage-gated Kᵥ channel, Ca²⁺-sensitive BK_{Ca} channels, and tandem-P-domain TASK channels), leading to membrane depolarization, activation of voltage-gated Ca²⁺ channels (VDCC), and release of neurotransmitters (see reviews). However, information of TRP channels in CB is scanty, but there are indications that they may participate in CB functions. Expressions of the canonical TRP (TRPC) channels have been detected in rat CB, CSN, and petrosal ganglion (PG). A non-selective cation conductance possesses pharmacological properties of TRP channels and responsive to low glucose has been recorded in rat glomus cells, and TRPV1 has been implicated for the increased CSN activity responding to thermo-stimulation. A recent study also showed that the elevation of [Ca²⁺]ᵢ in rat glomus cells induced by hypoxia, NaCN and FCCP was inhibited by 2-APB. The inventors examined the involvement of TRP channels in CB function, by first searching their published transcriptome data at GEO database, and found that multiple members of TRP (TRPC, TRPV and TRPM) subfamilies are expressed in mouse CB. These channels include Trpc1, Trp2, Trpc5, Trpc6, Trpm2, Trpm3, Trpm4, Trpm6, and Trpm7, Trpv1, Trpv2, and Trpv6 with Trpm7 being most abundantly expressed (FIG. 4A). The inventors then surveyed systemically the differential expression of TRP channels in CB, PG, superior cervical ganglion (SCG), and brainstem of C57BL/6J mouse using real-time PCR (qPCR). Among all TRPs, Trpm3, Trpm6 and Trpm7 expression were much higher in CB and PG, where Trpm7 expression was >15 times higher, compared to those in brainstem and SCG (FIG. 4B). Further comparison of Trpm3, Trpm6 and Trpm7 expression between the wildtype (WT), the leptin-deficient (ob/ob) and leptin receptor-null (db/db) mice showed that Trpm7 expression was significantly lower in CB and PG of both ob/ob and db/db mice. The remarkable (and surprising) preferential expression of Trpm7 channels in CB and PG, and its down-regulation in ob/ob and db/db mice identified in the present invention suggested that Trpm7 may be specific for the carotid chemosensitive afferent pathway and its expression is regulated by leptin and/or leptin-receptor mediated signaling pathways.

TRPM7 is an O₂-sensitive TRP channel. TRPM7 is a member of the melastatin-related TRP subfamily, and is characterized by its unique “chanzyme” structure comprising of an ion channel and an intracellular kinase domains. It is a non-selective cation channel permeates predominantly the divalent cations Ca²⁺ and Mg²⁺ under physiological conditions, and is the major channel for Mg²⁺ homeostasis. It has an outward rectifying voltage-current (I-V) relation with a prominent outward current carried by monovalent cations and a small inward ascribed to Ca²⁺ and Mg²⁺ influx. TRPM7 channel activity is regulated by PIP₂, phospholipase C¹⁴⁴, pH and reactive oxygen species (ROS). It can be inhibited by organic compounds including 2-APB, the aminobenzimidazole derivative NS8593 (also known as N-[(1R)-1,2,3,4-Tetrahydro-1-naphthalenyl]-1H-Benzimidazol-2-aminehydrochloride), carvacrol and sphingosine and its structural analog FTY720 (also known as fingolimod or 2-amino-2-[2-(4-octylphenyl)ethyl]propane-1,3-diol), and is activated by the small-molecule naltriben. More importantly, TRPM7 is recognized as an oxygen or redox-sensitive channel. TRPM7 plays a crucial role in ischemic neuronal death that is mediated by ROS. Hypoxia/anoxia also activates heterologously expressed TRPM7 in HEK293 cells. The present invention identifies The redox sensitivity of TRPM7, its preferential expression in mouse CB and PG (FIG. 4), the inhibition of hypoxia-activated CSN activity (FIG. 3) and Ca²⁺ response in glomus cells by 2-APB¹³⁷ that strongly suggest that TRPM7 may participate in CB chemoreception. Since TRPM7 is a Ca²⁺ permeating channel, its activation may enhance CB activity by increase Ca²⁺ influx and depolarize glomus cells to enhance neurotransmitter release. Moreover, TRPM7 has been shown to facilitate cholinergic vesicle fusion; hence it can participate in neurotransmitter transport and release, which is essential for CB chemoreception.

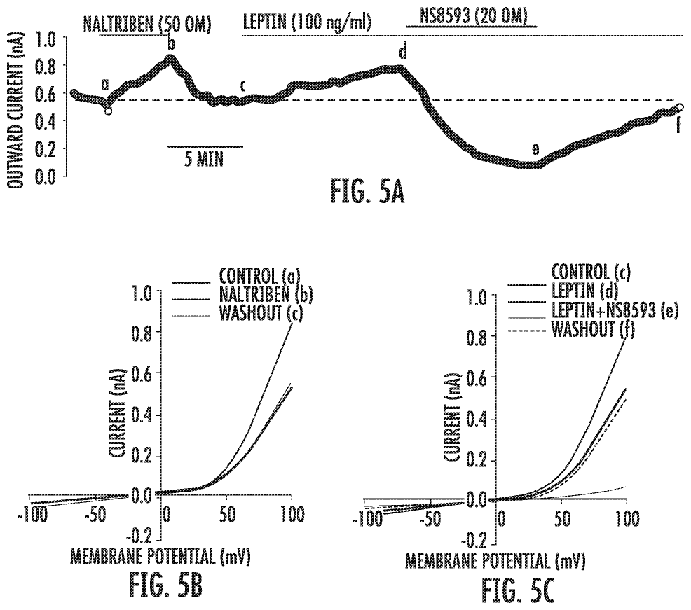

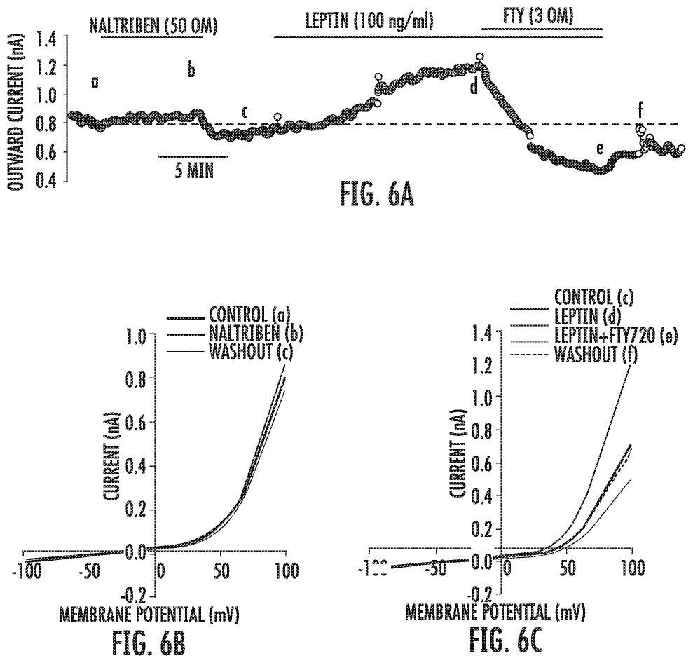

Leptin can regulate TRPM7 channel activity. Leptin exerts its action by binding to the long form of leptin receptors (LEPRb, ObRb), which is the only functional isoform of leptin receptor. The possible effect of leptin on TRPM7 channel activity was investigated, by using LEPRb expressing adrenal pheochromocytoma PC12 (PC12^{LEPRb}) cells as the cell model, because PC12 cells are oxygen-sensitive, express high level of TRPM7 and have been used widely in the studies of chemoreception. Non-selective cation current was recorded using amphotericin-B perforated-patch technique in the absence of extracellular and intracellular K⁺ (replaced by Cs⁺) and Mg²⁺, after VDCC was blocked by nifedipine and Cl⁻ currents were inhibited by DIDS and niflumic acid. Voltage-ramp of −100 to +100 mV elicited an outward rectifying current that resembled TRPM7 current reported in other cell types. Application of the TRPM7 agonist naltriben (50 μM) caused significant augmentation of the current, which was reversible upon naltriben washout (FIG. 5A, B). More importantly, leptin activated a similar increase of the current, which was almost completely abolished by the TRPM7 antagonists NS8593 and FTY120 (FIG. 5A, C; FIG. 6A, B). The leptin-induced current was not observed in PC12 cells transfected with a control plasmid (data not shown). The present invention provides the first evidence showing activation of leptin receptor is capable of enhancing TRPM7 activity, and it supports our hypothesis that leptin may enhance CB activity through regulation of TRPM7 activity.

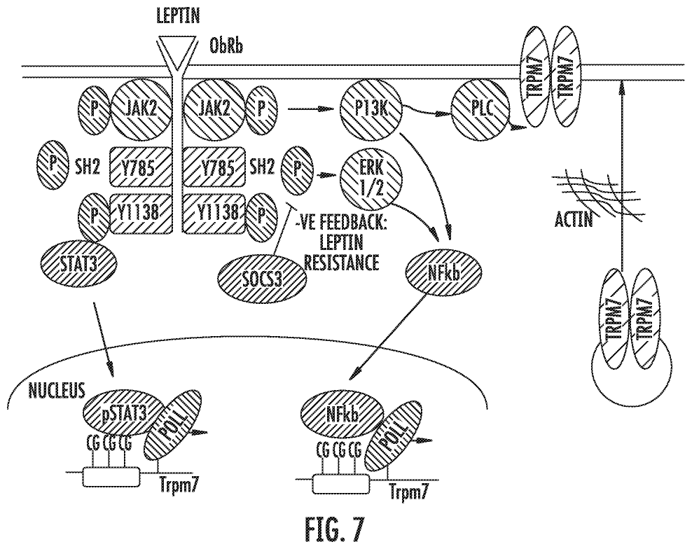

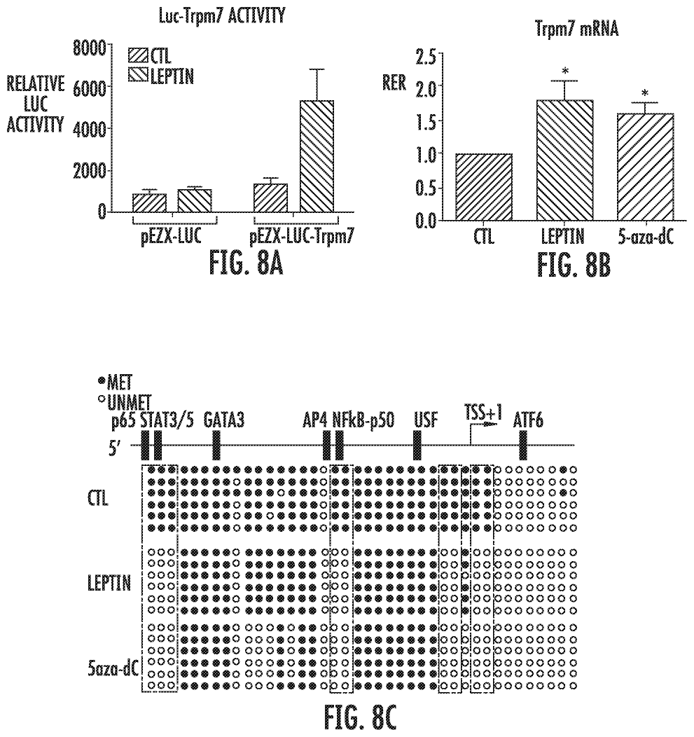

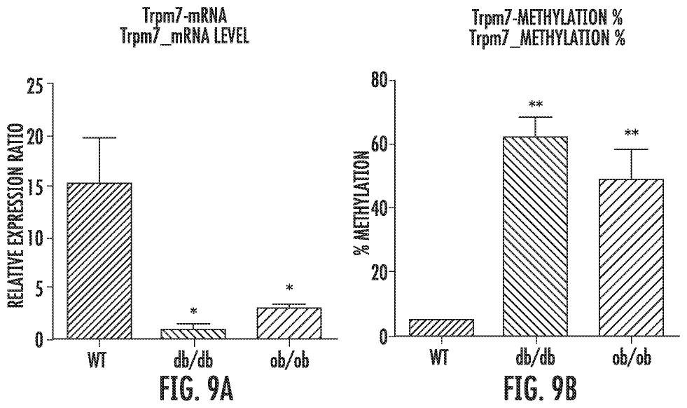

Leptin can regulate Trpm7 transcription To date, there are only a few studies demonstrating the regulation of Trpm7 transcription. The present invention identifies that Trpm7 expression is significantly lower in CB of ob/ob and db/db mice compared to WT (FIG. 4B) suggest that leptin may regulate Trpm7 expression in CB. Leptin can regulate gene transcription by binding to ObRb to activate Janus kinase 2/signal transducers and activator of transcription 3 (JAK2/STAT3) as well as ERK1/2 and PI3K pathways (FIG. 7), which are known to regulate a wide variety of genes. To examine the possible regulation of Trpm7 transcription by leptin, we searched the Trpm7 promoter sequence for potential binding sites for transcription factors (TFs). We found putative binding sites for TFs including STAT3, STAT5 and NFκB (p65 and p50) (FIG. 8C), which are downstream molecules of the ObRb signaling pathway. Furthermore, there are multiple CG dinucleotides (CpG) sites at the 5′ promoter region suggesting DNA methylation at specific CpG sites may alter TF binding to regulate Trpm7 transcription To examine the effect of leptin on Trpm7 transcription, PC12^{LEPRb} cells transfected with a pEZX-PG02 reporter or a pEZX-PG02 reporter with Trpm7 promoter inserted were exposed to leptin. The luciferase activity was significantly increased in cells treated with leptin for 48 hrs (FIG. 8A). The enhanced Trpm7 promoter activity was associated with significant increase in Trpm7 mRNA level (FIG. 8B), indicating that leptin indeed induces Trpm7 transcription. Leptin is known to alter gene-specific promoter methylation of metabolic genes. Given the fact that alteration in promoter methylation could modulate gene transcription, we postulate that Trpm7 promoter demethylation contributes to its upregulation. We measured the methylation status of Trpm7 by bisulfite sequencing (FIG. 8C) and found that CpG site-specific demethylation occurs in response to leptin exposure. Furthermore, these CpG sites coincide with the putative TF binding sites for STAT3/5 and NFkB (p50/p65). These findings suggest that demethylation at specific CpG sites may allow the binding of these potential TFs at Trpm7 promoter and activate Trpm7 transcription. Moreover, treatment of PC12^{LEPRb} cells with the DNA demethylation agent 5-aza-dC increased Trpm7, suggesting that Trpm7 expression is indeed regulated by DNA demethylation (FIGS. 8B and 8C). More importantly, leptin appears to regulate Trpm7 transcription in mouse CB via DNA methylation. Methylation of Trpm7 promoter is significantly higher in CB of ob/ob and db/db mice comparing to the WT control (FIG. 9B). This is consistent with the significantly lower Trpm7 mRNA expression in the CB of these mutant mice (FIG. 9A). Moreover, over-expression of ObRb-receptor in the carotid bodies of db/db mice enhances Trpm7 expression (see FIG. 19). Taken together, these results represent the first evidence indicating Trpm7 expression is regulated by the ObRb-mediated pathway, at least in part through DNA methylation.

In summary, the present invention identified that (1) ObRb is expressed in CB glomus cells, (2) leptin enhances hypoxia-induced CSN activity which can be blocked by TRP antagonists, (3) the O₂-sensitive Trpm7 gene is preferentially expressed in CB of WT but not the leptin or leptin-receptor deficient ob/ob or db/db mice, (4) leptin can acutely enhance TRPM7 current in PC12^{LEPRb} cells, and (5) treatment of PC12^{LEPRb} cells with leptin increases Trpm7 promoter activity and Trpm7 gene expression. The present invention demonstrates leptin can modulate CB activity acutely through activation of TRPM7 channel and chronically by upregulation of Trpm7 expression. Given that obesity is associated with hyperleptinemia, TRPM7 is a therapeutic target in such complications of obesity as sleep apnea and hypertension and inhibitors of TRPM 7 are likely therapeutic agents to prevent or treat these diseases in subjects.

### In Vivo Experiments

In vivo experiments have been performed demonstrating that leptin augments the CB chemoreflex via ObRb and that downstream TRPM7 channels exacerbate sleep apnea and induce hypertension. Human obesity predisposes to OSA, recurrent obstruction of the upper airway during sleep leading to intermittent hypoxia. Both obesity and OSA are associated with increased hypoxic sensitivity resulting in respiratory instability. Overly sensitive ventilatory response to the hypoxic stimulus termed a high loop gain causes central sleep apnea and aggravates OSA. Intermittent hypoxia was found to increase hypoxic sensitivity by perturbing the equilibrium between hypoxia inducible factors in the CB, but molecular mechanisms linking obesity to the CB chemoreflex have not been identified. CB express high levels of receptors for leptin, an adipocyte-produced hormone regulating metabolism and body weight. Human obesity also leads hyperleptinemia in proportion to the amount of adipose mass in the majority of individuals. Leptin levels are increased in patients with sleep apnea and rodents exposed to intermittent hypoxia, but the effects of leptin on the HVR and sleep apnea in diet-induced obesity (DIO) are unknown.

The augmented CB chemoreflex is a key mechanism of hypertension in OSA, which has been attributed to intermittent hypoxia. Given that the prevalence of OSA in obesity is greater than 50%, intermittent hypoxia-induced SNS activation is one of the major causes of hypertension in obesity. Obesity also increases the SNS activity independent of intermittent hypoxia and hypertension of obesity has been attributed to the CB activation. CB denervation treats hypertension in obese rats and CB silencing by hyperoxia or CB removal lowered blood pressure in humans. Obesity and OSA increase levels of leptin, which activates SNS increasing blood pressure. Leptin receptors are abundantly expressed in CB, but the role of leptin signaling in CB in hypertension is unknown.

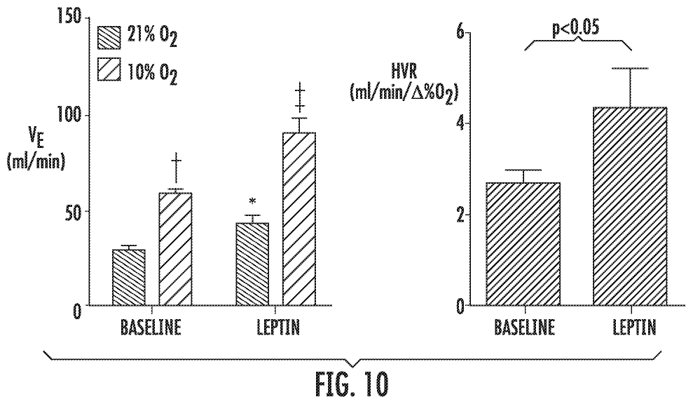

Leptin increases the HVR in lean mice. Minute ventilation (V_{E}) was measured at normoxic conditions and during 3-5 min exposure to 10% O₂ in awake lean male C57BL/6J mice, n=4, at baseline and during leptin administration (30 μg/day for 3 days via a subcutaneous osmotic pump). Serum leptin levels increased from 0.8±0.2 ng/ml to 33.2±1.9 ng/ml, which was similar to previously reported in DIO mice and obese humans, and then returned to the baseline. Mice decreased food intake and lost weight. Leptin increased V_{E} at baseline as a result of an increase in the metabolic rate (FIG. 10). Leptin increased the HVR by greater than 60%. Given that the HVR is regulated by the peripheral CB chemoreflex, the present invention demonstrates that leptin stimulates the CB chemoreflex.

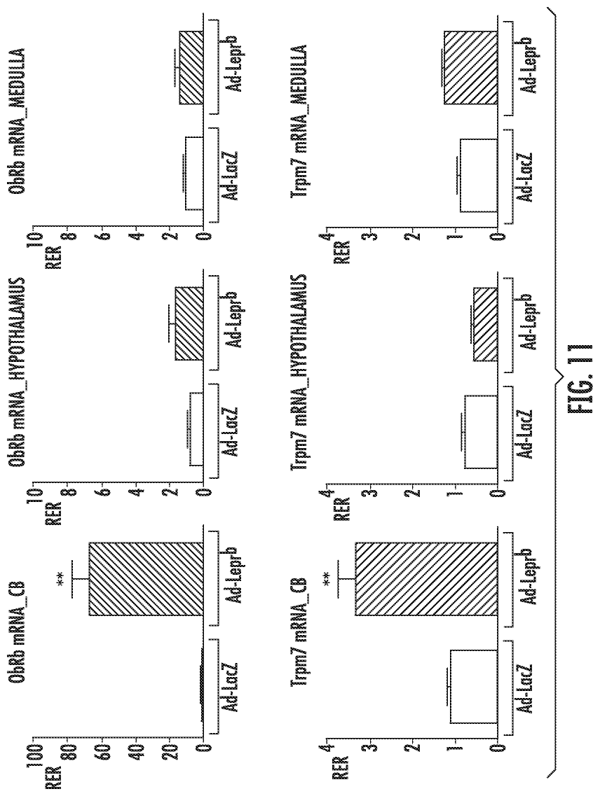

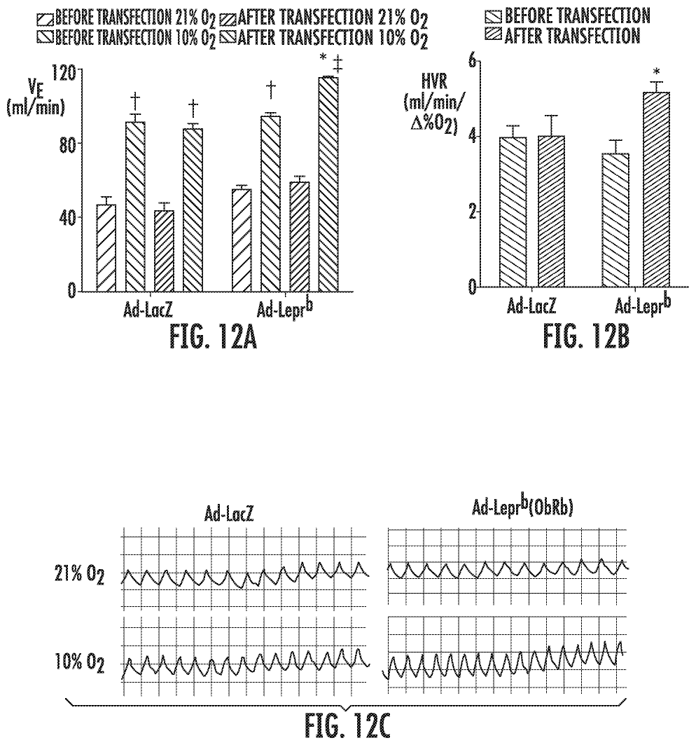

Selective leptin receptor overexpression in CB increases the HVR and Trpm7 gene expression in leptin receptor deficient db/db mice. As mentioned above, leptin regulates the HVR. The in vitro data showed that leptin may signal via Trpm7 to modulate the CB chemoreflex. However, studying the effects of leptin on CB in vivo poses a significant challenge due to the presence of ObRb receptors in multiple locations and a multitude of leptin effects. A unique novel model was developed to study effects of leptin selectively in the CB only by overexpressing the leptin ObRb receptor exclusively in CB of leptin receptor deficient db/db mice. Matrigel® containing Ad-Lepr^{b} adenovirus (the ObRb gene) or the control adenovirus Ad-LacZ (4.3×10¹² pfu/ml) graciously provided by Dr. Christopher Rhodes were applied to the CB areas bilaterally (n=8). After confirming that Matrigel® had solidified, the incision was closed. Ad-Lepr^{b} transfection significantly increased expression of both ObRb leptin receptor and Trpm7 in the CB (FIG. 11), but had no effect on ObRb and Trpm7 levels in the hypothalamus and medulla. db/db mice maintained their food intake and body temperature and did not lose weight. As expected, serum leptin levels were extremely high and unchanged after the transfection, 61.8±5.1 ng/ml. Transfection did not change room air V_{E} (FIG. 12). In contrast, ObRb expression in the CB resulted in acute hypoxic hyperventilation (FIG. 12A) and increased HVR (FIG. 12B), which did not occur in the control Ad-LacZ group. Our data suggest that leptin regulates Trpm7 expression and stimulates the HVR acting via the ObRb receptor in the carotid bodies. Next we explored whether an increase in the HVR will induce respiratory instability and sleep apnea in db/db mice.

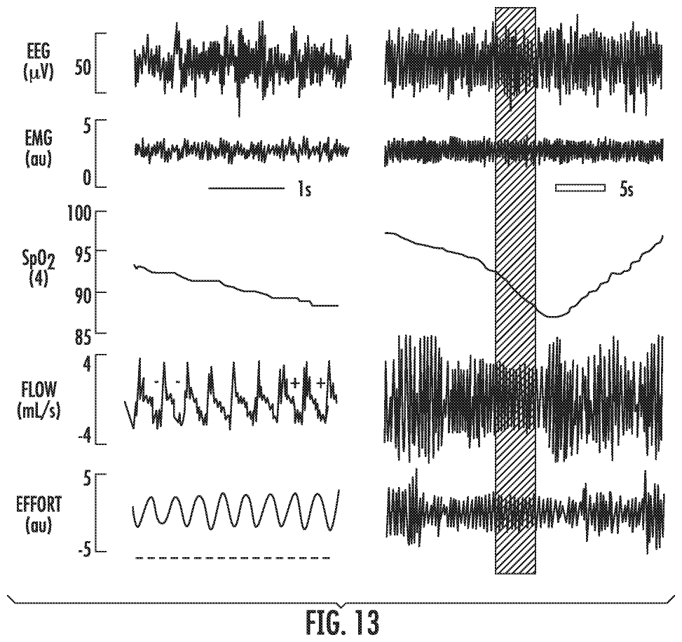

db/db and DIO mice have obstructive sleep apnea. A novel plethysmographic method was developed and validated. Obstruction was characterized by the development of inspiratory airflow limitation (IFL) during sleep, the cardinal feature of apnea-hypopnea syndrome with continuing effort. We demonstrated that db/db mice (n=4) and DIO (n=4) have obstructive sleep apnea as manifested by IFL with oxyhemoglobin desaturations (FIG. 13). Sleep apnea severity was quantified by: 1) the oxygen desaturation index (ODI), desaturations ≥3% from baseline; 2) the apnea-hypopnea index (AHI) defined as the ODI events accompanied by reductions in airflow. Obstructive from central apneas was separately characterized by a decrease in effort and the absence of IFL. Central apneas have been described in C57BL/6J mice and can be increased by leptin. Thus, leptin-resistant db/db and DIO mice show evidence of obstructive sleep apnea identical to humans.

ObRb receptor overexpression in the CB increases sleep apnea in db/db mice. Baseline sleep studies were recorded in 4 db/db mice and then two mice were transfected to the CB with Ad-Lepr^{b} and two others with a control virus Ad-LacZ. Sleep studies were repeated on Day 9 after transfection. All mice had occasional hypopneas at baseline with the total ODI of 4.0±1.1/hr, including 2.2±0.7/hr in NREM and 34.0±7.7/hr in REM sleep. ObRb receptor transfection increased the ODI to 11.5±2.4/hr predominantly in NREM sleep (9.3±0.5/hr). In contrast, the severity of sleep apnea did not change in the control group. Thus, an increase in hypoxic sensitivity mediated by ObRb signaling in the CB may exacerbate sleep apnea in obesity.

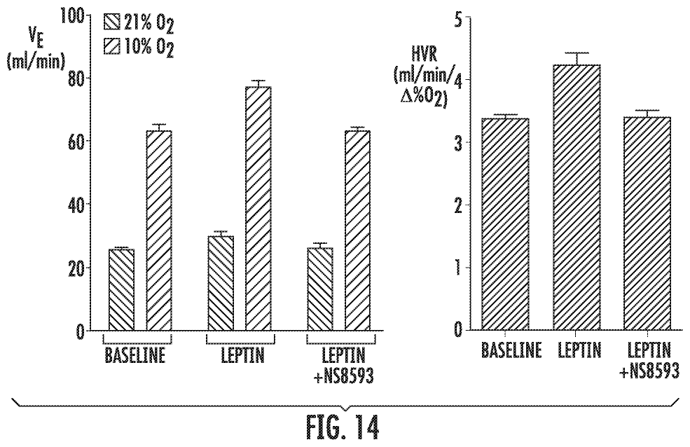

Trpm7 inhibition prevents effects of leptin on the HVR. Our Preliminary Data in cell culture and with the isolated carotid sinus nerve suggests that leptin signals in CB via the Trpm7 channel. Male C57BL/6J mice (n=2) were treated with leptin for 3 days as described above. After 5 day recovery, leptin and a TRPM7 blocker NS8593 (10 mg/kg/day) were administered subcutaneously via an osmotic pump for 3 days. Mice reduced food intake and lost weight with leptin, regardless of the TRPM7 inhibitor. In contrast, NS8593 prevented leptin-induced hypoxic hyperventilation abolishing leptin-induced increase in the HVR without any effect on basal ventilation (FIG. 14). The present invention suggest that leptin's effect on the HVR in vivo is mediated via TRPM7.

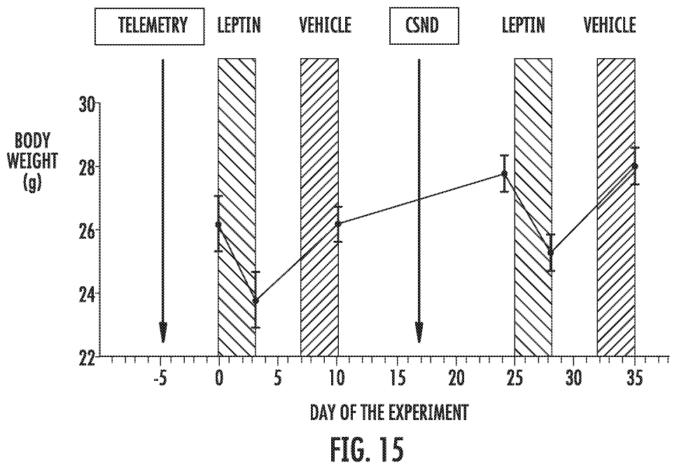

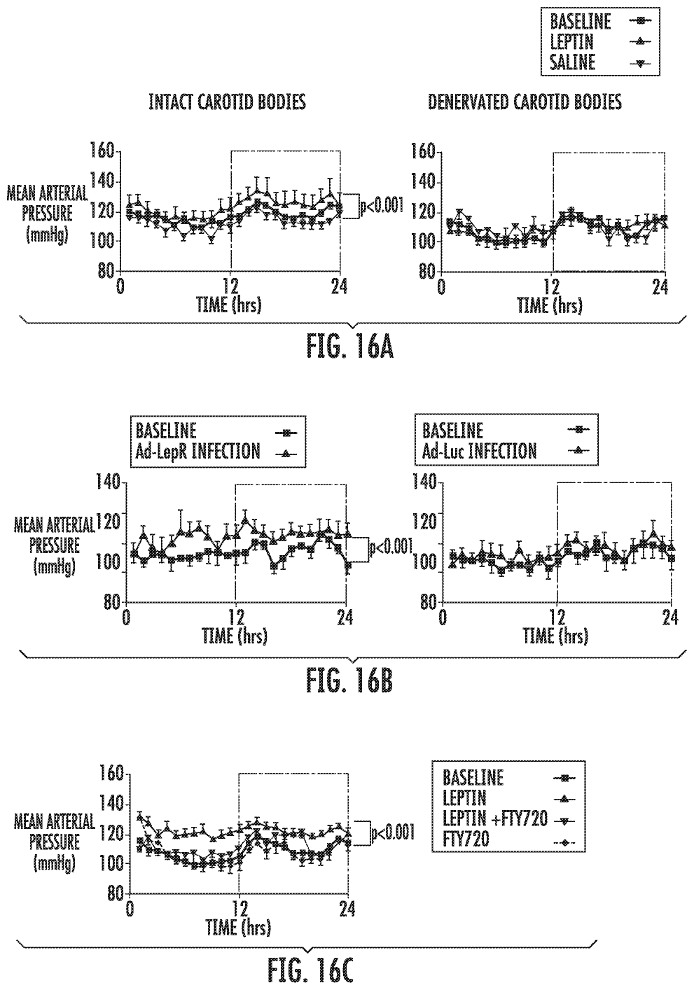

Leptin signals in the CB to increase blood pressure. Male C57BL/6J mice, n=6 housed in a 12 hr light/dark cycle underwent telemetry implantation (Data Sciences) in the left femoral artery as we have described. After 5 day recovery, mice have been treated with leptin for 3 days as described above, which resulted in expected weight loss (FIG. 15). After 4 day recovery mice were treated with vehicle (saline) regaining weight. On Day 17^{th} of the protocol, carotid sinus nerve dissection (CSND, CB denervation) was performed bilaterally. After 1 week recovery, leptin and vehicle infusions were repeated. CSND did not prevent weight loss during leptin infusion suggesting that the CBs are not implicated in metabolic effects of leptin. In contrast, CB denervation greatly modified cardiovascular effects of leptin. Indeed, in mice with intact carotid bodies leptin increased mean arterial pressure by 13 mm Hg during the day and by 16 mm Hg at night (FIG. 16A). The effects of leptin were completely abolished by CB denervation. Thus, leptin increases blood pressure acting in the CB.

Leptin receptor (LepR) transfection to carotid body causes hypertension in leptin receptor deficient db/db mice. Telemetry was implanted in 12 db/db mice as described above. Blood pressure was measured at baseline. Matrigel® containing Ad-Lepr^{b} adenovirus (the ObRb gene) or the control adenovirus Ad-Luc (4.3×10¹² pfu/ml) graciously provided by Dr. Christopher Rhodes were applied to the CB areas bilaterally (n=6 per group). The incision was closed after confirming that Matrigel® had solidified. 15 days after transfection blood pressure was measured again. LepR transfection to carotid body increased daytime mean arterial pressure by 8.7±3.1 mm Hg and nighttime mean arterial blood pressure by 9.8±2.9 mm Hg (FIG. 16B, p<0.05). This experiment provided another evidence that leptin acts on ObRb receptors in the carotid bodies to increase blood pressure.

FTY720 abolishes leptin-induced hypertension. There are multiple TRPM7 inhibitors available. We used a potent TRPM7 inhibitor FTY720 for several reasons. FTY720 is a novel immunomodulator and a structural homolog of shingosine-1-phosphate (S1P) blocking S1P effects in many diseases, including atherosclerosis, cancer, asthma, autoimmune diseases and acute lung injury. First, FTY270 has already been approved by FDA for multiple sclerosis (fingolimod). Second, FTY720 has a long half-life of 19-28 hrs in mice and 7 days in humans. The experiment was performed in lean (24-26 g) C57BL/6J mice (n=5). Telemetry for continuous blood pressure recording was inserted. After 5 day recovery, mice were treated with leptin for 3 days as above. The pump was removed. After 7 day recovery, leptin was reinfused as above, but FTY720 was infused SC simultaneously at 0.1 mg/day (approximately 3 mg/kg/day). Systemic administration of FTY720 completely abolished leptin-induced hypertension (FIG. 16C). The present invention demonstrates: 1) systemic leptin administration increases the HVR and this increase is attenuated by a TRPM7 blocker; and 2) selective ObRb receptor expression in the CB up-regulates Trpm7 gene expression in the CB that increases the HVR and exacerbates sleep apnea in leptin receptor deficient db/db mice. The present invention suggests that leptin increases the HVR and aggravates sleep apnea acting via ObRb receptors and downstream Trpm7 channels in the CB.

The present invention demonstrating: (1) leptin increases blood pressure and this increase is prevented by CB denervation (FIG. 16A), (2) leptin increases CSN activity and this increase is abolished by TRP channel inhibitors (FIG. 3); (3) TRPM7 is the most abundant TRP channel in CB of WT mice, but not in leptin or leptin-receptor deficient mice (FIG. 4); (4) leptin induces TRPM7 current in PC12 cells (FIGS. 5 and 6); (5) leptin regulates Trpm7 methylation, promoter activity, and gene expression in CB (FIGS. 8 and 9); (6) Ob expression in CB of leptin receptor deficient db/db mice increases Trpm7 expression (FIG. 11); and (7) TRPM7 blocker prevents a leptin-induced increase in the CB chemoreflex (FIGS. 14); and (8) Leptin-induced hypertension is abolished by administration of a TRPM7 blocker FTY720 (FIG. 16C). The present invention illustrates that leptin causes hypertension in obesity acting via ObRb receptors and downstream Trpm7 channels in the CB suggesting TRPM7 inhibitors be used in novel methods of preventing and treating hypertension in patients, preferably severe obesity patients.

### Mechanism of Leptin and Transient Receptor Potential (TRP) Channels in the Carotid Body for Hypertension Control.

Obesity leads to cardiovascular morbidity and mortality acting via multiple mechanisms including hypertension and obstructive sleep apnea. High levels of leptin have been associated with both conditions. The carotid bodies (CB) express leptin receptor (LepR), but the mechanism of leptin in CB is unknown. The inventors demonstrate that hyperleptinemia increases the CB chemoreflex leading to hypertension by examining leptin's effect in carotid body, male C57BL/6J mice (n=6) that were implanted with telemetry in the left femoral artery for blood pressure monitoring and then recording at baseline, during leptin infusion (120 ug/day for 3 days via a SC pump) before and after carotid sinus nerve dissection. (CSND). In mice with intact CB, leptin increased mean arterial pressure by 13 mm Hg during the day and by 16 mm Hg at night (p=0.003 for the effect of leptin). CSND completely abolished leptin-induced hypertension (p<0.001 for the effect of CSND). Leptin receptor expression in carotid bodies of leptin receptor deficient db/db mice led to the same effect (n=6, p<0.05). The inventors performed RNA microarrays in CB and found that Trpm7 was highly expressed in the carotid body (FIG. 4A). Finally, the inventors tested the effect of a Trpm7 inhibitor on leptin-induced hypertension. The inventors performed blood pressure monitoring in male C57BL/6J mice (n=5˜9) at baseline, during leptin infusion with or without Trpm7 inhibitor, FTY720 (3 mg/kg/day, sc, n=5) for 24 hrs. The inventors found that leptin-induced hypertension was prevented by FTY720 treatment. The inventors conclude that leptin increases blood pressure acting in the CB via the Trpm7 channel.

Obesity is a leading cause of high cardiovascular morbidity and mortality. It is characterized by an increased leptin level, which has been implicated in increased sympathetic activity and the pathogenesis of hypertension. However, the mechanism by which leptin enhances sympathetic activity is unclear. Leptin receptors (LEPRs) are expressed in the carotid bodies, the most important arterial chemoreceptors for the cardiovascular-respiratory chemoreflex. Since leptin is known to regulate many different ion channels, we hypothesize that leptin may exert its effects through modulation of TRPM7 channel activity. To test this hypothesis, the inventors used LEPR (LEPRb) expressing pheochromocytoma (PC12^{LEPRb}) cells, which expresses high levels of TRPM7 channels, as the cell model. Non-selective cation channels were recorded under amphotericin-B perforated-patch techniques with K⁺ replaced by Cs⁺ in the presence of voltage-gated Ca²⁺ channel and Cl⁻ channel blockers. Voltage-ramp from −100 to 100 mV activated an outward-rectifying current. Removal of extracellular divalent ions enhanced the inward current, consistent with the inward divalent ion selectivity of TRPM7. The specific TRPM7 agonist naltriben caused significant increase of the current, which was abolished by the TRPM7 antagonist NS8593 and FTY720, indicating that functional TRPM7 channels are present in PC12^{LEPRb} cells. More importantly, leptin at concentration of 10-100 ng/ml caused concentration-dependent increase in the outward rectifying current. The enhanced current was completely blocked by NS8593 and FTY720. These results for the first time demonstrated that stimulation of leptin receptor is capable of activating TRPM7 channels, and suggest that leptin may exert its physiological effects through modulation of TRPM7 activity.

Embodiments of the disclosure concern methods and/or compositions for treating and/or preventing respiratory disease, cardiovascular disease, or both in which modulation of TRPM7 is directly or indirectly related. In certain embodiments, individuals with a respiratory disease, cardiovascular disease, or both are treated with a modulator of the pathway, and in specific embodiments an individual with sleep apnea, central apnea, Cheyne-Stokes respiration and/or hypertension is provided a modulator of TRPM7, such as an inhibitor of TRPM7.

In certain embodiments, the level to which an inhibitor of TRPM7 decreases TRPM7 activity may be any level so long as it provides amelioration of at least one symptom of the neurological disorder, including sleep apnea and/or hypertension, as examples. The level of activity may decrease by at least 2, 3, 4, 5, 10, 25, 50, 100, 1000, or more fold expression compared to the level of expression in a standard, in at least some cases.

An individual known to have sleep apnea and/or hypertension, suspected of having sleep apnea and/or hypertension, or at risk for having sleep apnea and/or hypertension may be provided an effective amount of an inhibitor of TRPM7 including 2-APB and/or SKF-96365, as examples. Those at risk for a respiratory disease and/or cardiovascular disease may be those individuals having one or more genetic factors, may be of advancing age, and/or may have a family history, for example.

In particular embodiments of the disclosure, an individual is given an agent for respiratory and/or cardiovascular therapy in addition to the one or more inhibitors of TRPM7. When combination therapy is employed with one or more inhibitors of TRPM7, the additional therapy may be given prior to, at the same time as, and/or subsequent to the one or more inhibitors of TRPM7.

### Pharmaceutical Preparations

Pharmaceutical compositions of the present invention comprise an effective amount of one or more inducers of expression of peroxisome proliferator-activated receptor-□ coactivator-inhibitor of TRPM7, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one or more inhibitors of TRPM1 or additional active ingredient will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The one or more inhibitors of TRPM7 may comprise different types of carriers depending on whether it is to be administered in solid, liquid or aerosol form, and whether it need to be sterile for such routes of administration as injection. The present compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The one or more inhibitors of TRPM7 may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include one or more inhibitors of TRPM7 one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the one or more inhibitors of TRPM7 may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Alimentary Compositions and Formulations

In one embodiment of the present disclosure, the one or more inhibitors of TRPM7 are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally- administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### Parenteral Compositions and Formulations

In further embodiments, one or more inhibitors of TRPM7 may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### Miscellaneous Pharmaceutical Compositions and Formulations

In other preferred embodiments of the invention, the active compound one or more inhibitors of TRPM7 may be formulated for administration via various miscellaneous routes, for example, topical (i.e., transdermal) administration, mucosal administration (intranasal, vaginal, etc.) and/or inhalation. Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### Kits of the Disclosure

Any of the compositions described herein may be comprised in a kit. In a non-limiting example, one or more inhibitors of TRPM7 may be comprised in a kit.

The kits may comprise a suitably aliquoted one or more inhibitors of TRPM7 and, in some cases, one or more additional agents. The component(s) of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the one or more inhibitors of TRPM7 and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The one or more inhibitors of TRPM7 composition(s) may be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of preventing or treating a respiratory disease, a cardiovascular disease or both in an obese subject comprising administering to the obese subject an effective amount of TRPM7 inhibitor or a pharmaceutically acceptable salt, solvate, or stereoisomer thereof and treating or preventing the respiratory disease, the cardiovascular disease or both in the obese subject.

2. The method of claim 1 wherein the respiratory disease is sleep apnea, central apnea, or Cheyne-Stokes respiration.

3. The method of claim 2 wherein the sleep apnea is obstructive sleep apnea.

4. The method of claim 1 wherein the cardiovascular disease is hypertension or heart failure.

5. The method of claim 1 wherein the subject is human.

6. The method of claim 1 wherein the effective amount of TRPM7 inhibitor is a pharmaceutical composition comprising a pharmaceutically acceptable carrier.

7. The method of claim 1 wherein the TRPM7 inhibitors are selected from the group consisting of 2-APB 174, Spermine, SKF-96365, Nafamostat, Carvacrol, NDGA, AA861, MK886, Waixenicin A, NS8593, Quinine, CyPPA, Dequalinium, SKA31, UCL 1684, Sphingosine, fingolimod, and combinations thereof.

8. The method of claim 1 wherein the TRPM7 inhibitor is fingolimod.

9. A method of preventing or treating a cardiovascular disease in a subject comprising administering to the subject an effective amount of TRPM7 inhibitor or a pharmaceutically acceptable salt, solvate, or stereoisomer thereof and preventing or treating the cardiovascular disease in the subject.

10. The method of claim 9 wherein the cardiovascular disease is hypertension.

11. The method of claim 9 wherein the subject is human.

12. The method of claim 9 wherein the subject is obese

13. The method of claim 9 wherein the effective amount of TRPM7 inhibitor is a pharmaceutical composition comprising a pharmaceutically acceptable carrier.

14. The method of claim 9 wherein the TRPM7 inhibitors are selected from the group consisting of 2-APB 174, Spermine, SKF-96365, Nafamostat, Carvacrol, NDGA, AA861, MK886, Waixenicin A, NS8593, Quinine, CyPPA, Dequalinium, SKA31, UCL 1684, Sphingosine, fingolimod and combinations thereof.

15. A method of preventing or treating a respiratory disease in a subject comprising administering to the subject an effective amount of TRPM7 inhibitor or a pharmaceutically acceptable salt, solvate, or stereoisomer thereof and preventing or treating the respiratory disease in the subject.

16. The method of claim 15 wherein the respiratory disease is sleep apnea.

17. The method of claim 15 wherein the sleep apnea is obstructive sleep apnea.

18. The method of claim 15 wherein the subject is obese.

19. The method of claim 15 wherein the effective amount of TRPM7 inhibitor is a pharmaceutical composition comprising a pharmaceutically acceptable carrier.

20. The method of claim 15 wherein the TRPM7 inhibitors are selected from the group consisting of 2-APB 174, Spermine, SKF-96365, Nafamostat, Carvacrol, NDGA, AA861, MK886, Waixenicin A, NS8593, Quinine, CyPPA, Dequalinium, SKA31, UCL 1684, Sphingosine, fingolimod, and combinations thereof.
